SEQUENCE LISTING (28 sequences)

SEQ ID NO: 1 - RNA, 22 nt, Homo sapiens
  source 1..22: mol_type = other RNA
tctgtgcttcacccctacccag

SEQ ID NO: 2 - RNA, 20 nt, Mus musculus
  source 1..20: mol_type = other RNA
tttctgacctggatccccag

SEQ ID NO: 3 - RNA, 21 nt, Mus musculus
  source 1..21: mol_type = other RNA
tgtgtccccttcctctcacag

SEQ ID NO: 4 - RNA, 24 nt, Mus musculus
  source 1..24: mol_type = other RNA
tatgggtggctggtctcagaagag

SEQ ID NO: 5 - RNA, 22 nt, Mus musculus
  source 1..22: mol_type = other RNA
tgaggaatcctgatctctcgcc

SEQ ID NO: 6 - RNA, 21 nt, Mus musculus
  source 1..21: mol_type = other RNA
tggttatccctgtcctcttcg

SEQ ID NO: 7 - DNA, 19 bp, Mus musculus
  source 1..19: mol_type = other DNA
catcggcattttgaacgag

SEQ ID NO: 8 - DNA, 20 bp, Mus musculus
  source 1..20: mol_type = other DNA
cgagctcactctctgtggtg

SEQ ID NO: 9 - DNA, 23 bp, Mus musculus
  source 1..23: mol_type = other DNA
cctctgacccttaaggagcttat

SEQ ID NO: 10 - DNA, 18 bp, Mus musculus
  source 1..18: mol_type = other DNA
cgttgcacaggggagtct

SEQ ID NO: 11 - DNA, 20 bp, Mus musculus
  source 1..20: mol_type = other DNA
agactccagccacactccaa

SEQ ID NO: 12 - DNA, 18 bp, Mus musculus
  source 1..18: mol_type = other DNA
tgacagcgcagctcattg

SEQ ID NO: 13 - DNA, 26 bp, Mus musculus
  source 1..26: mol_type = other DNA
aaaatcatccaaaagatactgaacaa

SEQ ID NO: 14 - DNA, 20 bp, Mus musculus
  source 1..20: mol_type = other DNA
ctttggttcttccgttgagg

SEQ ID NO: 15 - DNA, 20 bp, Mus musculus
  source 1..20: mol_type = other DNA
gacacattgagcatccaagg

SEQ ID NO: 16 - DNA, 27 bp, Mus musculus
  source 1..27: mol_type = other DNA
aacagattggtcattgtatgtactcag

SEQ ID NO: 17 - DNA, 25 bp, Mus musculus
  source 1..25: mol_type = other DNA
gctaccaaactggatataatcagga

SEQ ID NO: 18 - DNA, 24 bp, Mus musculus
  source 1..24: mol_type = other DNA
ccaggtagctatggtactccagaa

SEQ ID NO: 19 - DNA, 19 bp, Mus musculus
  source 1..19: mol_type = other DNA
agttgacggaccccaaaag

SEQ ID NO: 20 - DNA, 20 bp, Mus musculus
  source 1..20: mol_type = other DNA
agctggatgctctcatcagg

SEQ ID NO: 21 - DNA, 21 bp, Mus musculus
  source 1..21: mol_type = other DNA
tcttctcattcctgcttgtgg

SEQ ID NO: 22 - DNA, 20 bp, Mus musculus
  source 1..20: mol_type = other DNA
gaggccatttgggaacttct

SEQ ID NO: 23 - RNA, 22 nt, Mus musculus
  source 1..22: mol_type = other RNA
tctgmgctrcacccctacccag

SEQ ID NO: 24 - RNA, 20 nt, Peucedanum sp.
  source 1..20: mol_type = other RNA
tttctgagctgcatccccag

SEQ ID NO: 25 - RNA, 21 nt, Peucedanum sp.
  source 1..21: mol_type = other RNA
tgtgtccccatcgtctcacag

SEQ ID NO: 26 - RNA, 24 nt, Peucedanum sp.
  source 1..24: mol_type = other RNA
taagggtggctgttctcagaagag

SEQ ID NO: 27 - RNA, 22 nt, Peucedanum sp.
  source 1..22: mol_type = other RNA
aggggaatcctgatctctcgcc

SEQ ID NO: 28 - RNA, 21 nt, Peucedanum sp.
  source 1..21: mol_type = other RNA
tggttatccctcccctcttcg

(19) United States
(12) Patent Application Publication
(10) Pub. No.: US 2025/0009827 A1
(43) Pub. Date: Jan. 9, 2025

(54) PHARMACEUTICAL, FOOD, AND COSMETIC COMPOSITIONS AND PRODUCTS

(71) Applicants: THE UNIVERSITY OF TOKYO, Tokyo (JP); IDO MEDICAL CO., LTD., Tokyo (JP)

(72) Inventors: Tetsu AKIYAMA, Tokyo (JP); Yuki KAMOSHIDA, Tokyo (JP); Tomoatsu HAYASHI, Tokyo (JP); Yusuke YAMAZUMI, Tokyo (JP); Takeaki ODA, Tokyo (JP); Mima SHIKANAI, Tokyo (JP); Yasunari KAGEYAMA, Tokyo (JP); Takuya KOJIMA, Tokyo (JP)

(73) Assignees: THE UNIVERSITY OF TOKYO, Tokyo (JP); IDO MEDICAL CO., LTD., Tokyo (JP)

(21) Appl. No.: 18/588,968

(22) Filed: Feb. 27, 2024

(30) Foreign Application Priority Data
Feb. 28, 2023 (JP) 2023-030573

(51) Int. Cl. A61K 36/23; A61P 3/10; A61P 29/00

(52) U.S. Cl. CPC A61K 36/23; A61P 3/10; A61P 29/00

(57) ABSTRACT

A pharmaceutical composition comprising an effective amount of extracellular vesicles derived from a plant. A food composition comprising an effective amount of extracellular vesicles derived from a plant. A cosmetic composition comprising an effective amount of extracellular vesicles derived from a plant. For example, the plant is selected from the group consisting of coastal hog fennel, Madeira vine, lemongrass, green garlic, turmeric, crimson glory vine, and bitter melon.

### SEQUENCE LISTING

The instant application contains a Sequence Listing in electronic format which has been submitted via EFS-Web. Said Sequence Listing, created on Feb. 5, 2024, is named “4610US-TU_Sequence Listing.xml” and is 25,375 bytes in size. The information in the electronic format of the Sequence Listing is part of the present application and is incorporated herein by reference in its entirety.

### TECHNICAL FIELD

The present invention relates to pharmaceutical, food, and cosmetic compositions and products.

### BACKGROUND

Extracellular vesicles (EVs) are vesicles with a diameter in the range of 30-1000 nm, including various vesicles of different origin, size, or function. Recently, various studies are being conducted to use extracellular vesicles as therapeutic agents and biomarkers (Non patent document 1).

[Non patent document] “Molecular evaluation of five different isolation methods for extracellular vesicles reveals different clinical applicability and subcellular origin” RE Veerman et al. J Extracell Vesicles. 2021 10: e12128

### SUMMARY OF THE INVENTION

An object of the present invention is to provide novel pharmaceutical, food, and cosmetic compositions and products.

An aspect of the present invention is a pharmaceutical composition or product comprising extracellular vesicles derived from a plant as an active ingredient.

The pharmaceutical product may be an anti-inflammatory agent, a muscle strength enhancer, a therapeutic agent for diabetes, a prophylactic agent for diabetes, an agent for reducing subcutaneous fat, a slimming agent, a therapeutic agent for dry eye syndrome, a therapeutic agent for irritable bowel syndrome, a cytokine storm inhibitor, a therapeutic agent for coronavirus infection, or a therapeutic agent for dementia. The anti-inflammatory agent may be a therapeutic agent for encephalitis, myalgic encephalomyelitis, chronic fatigue syndrome, post-coronavirus diseases, inflammatory bowel diseases, or dermatitis. The pharmaceutical product may be in the form of ointments, oral medicaments, eye drops, nasal drops, inhalants, or injections. The extracellular vesicles may have an average particle diameter in the range of 125-155 nm, 135-165 nm, 110-140 nm, or 160-190 nm, and more preferably, 135-145 nm, 145-155 nm, 120-130 nm, or 170-180 nm. Here, 90% or more, or 95% or more of the extracellular vesicles may have a particle diameter in the range of 50-250 nm.

Another aspect of the present invention is a food composition or product comprising extracellular vesicles derived from a plant as an active ingredient. The extracellular vesicles may have an average particle diameter in the range of 125-155 nm or 135-145 nm. Here, 90% or more, or 95% or more of the extracellular vesicles may have a particle diameter in the range of 50-250 nm.

Another aspect of the present invention is a cosmetic composition or product comprising extracellular vesicles derived from a plant as an active ingredient. The extracellular vesicles may have an average particle diameter in the range of 125-155 nm or 135-145 nm. Here, 90% or more, or 95% or more of the extracellular vesicles may have a particle diameter in the range of 50-250 nm.

Another aspect of the present invention is a suppressor of expression of an inflammatory cytokine, the suppressor comprising extracellular vesicles derived from a plant as an active ingredient. The inflammatory cytokine may be IL-6, IL-1β, CXCL2, or IL-4.

Yet another aspect of the present invention is a suppressor of expression of a senescence marker, the suppressor comprising extracellular vesicles derived from a plant as an active ingredient. The senescence marker may be p15 or p16.

The plant may be selected from the group consisting of coastal hog fennel, Madeira vine, lemongrass, green garlic, turmeric, crimson glory vine, and bitter melon.

Still another aspect of the present invention is pharmaceutical, food, and cosmetic compositions and products and a suppressor of expression of an inflammatory cytokine, comprising RNA, as an active ingredient, the RNA having one of the following nucleotide sequences.

hsa-miR-6737-3p:
(SEQ ID NO: 1)
UCUGUGCUUCACCCCUACCCAG;
mmu-miR-7006-3p:
(SEQ ID NO: 2)
UUUCUGACCUGGAUCCCCAG;
mmu-miR-6995-3p:
(SEQ ID NO: 3)
UGUGUCCCCUUCCUCUCACAG;
mmu-miR-12178-5p:
(SEQ ID NO: 4)
UAUGGGUGGCUGGUCUCAGAAGAG;
mmu-miR-3063-3p:
(SEQ ID NO: 5)
UGAGGAAUCCUGAUCUCUCGCC;
and
mmu-miR-487b-5p:
(SEQ ID NO: 6)
UGGUUAUCCCUGUCCUCUUCG.

The inflammatory cytokine may be IL-6.

The present invention made it possible to provide novel pharmaceutical, food, and cosmetic compositions and products.

### BRIEF DESCRIPTION OF THE DRAWINGS

FIG. 1A is a photograph showing fractions in a centrifuge tube, which were obtained by centrifuging juice from coastal hog fennel, suspending the resulting precipitants in PBS and fractionating the suspension by sucrose density gradient ultracentrifugation; FIG. 1B is a graph showing a particle size distribution of extracellular vesicles contained in a band (the second band) at densities of 1.12-1.18 g/mL; and FIG. 1C is a graph showing an average particle diameter of the extracellular vesicles contained in the second band, in an example of the present invention.

FIG. 2 is a figure showing that extracellular vesicles derived from coastal hog fennel have effects of suppressing the LPS-stimulated induction of expression of inflammatory cytokines in experiments in vitro using cultured cells, in an example of the present invention.

FIGS. 3A-3B are figures showing that extracellular vesicles derived from coastal hog fennel have an effect of suppressing bleeding in mouse model of colitis, in an example of the present invention.

FIG. 4 is a figure showing that extracellular vesicles derived from coastal hog fennel have an anti-inflammatory effect on epithelium tissues of mouse model of colitis, in an example of the present invention.

FIG. 5 is a figure showing that extracellular vesicles derived from coastal hog fennel suppress the induction of senescence markers by the induction of expression of Ras gene, in an example of the present invention.

FIGS. 6A-6B are figures showing that extracellular vesicles derived from coastal hog fennel have an effect of enhancing muscle strength in mice, in an example of the present invention.

FIGS. 7A-7C are figures showing that extracellular vesicles derived from coastal hog fennel have FIG. 7A an effect of reducing insulin resistance: FIG. 7B an effect of suppressing the fat accumulation; and FIG. 7C an effect of reducing HbA1c concentrations, due to high-fat diets fed to mice in an example of the present invention.

FIGS. 8A-8D are figures showing that extracellular vesicles derived from coastal hog fennel have an effect of alleviating symptoms of dry eye syndrome, in an example of the present invention.

FIG. 9 is a figure showing that extracellular vesicles derived from coastal hog fennel have an effect of reducing swelling of ears associated with inflammation of skin, in an example of the present invention.

FIG. 10 is a figure showing that extracellular vesicles derived from coastal hog fennel have an effect of suppressing skin inflammation-associated infiltration of immune cells, in an example of the present invention.

FIG. 11 is a figure showing that extracellular vesicles derived from coastal hog fennel have an effect of suppressing tissue thickening due to ear swelling associated with inflammation of skin, in an example of the present invention.

FIG. 12 is a figure showing that extracellular vesicles derived from coastal hog fennel have an effect of suppressing skin inflammation-associated expression of inflammatory factors, in an example of the present invention.

FIG. 13 is a figure showing that extracellular vesicles prepared from coastal hog fennel, Madeira vine, lemongrass, green garlic, turmeric, crimson glory vine, and bitter melon have an effect of suppressing the induction of LPS-stimulated expression of inflammatory cytokines in experiments in vitro using cultured cells, in an example of the present invention.

FIGS. 14A-14B are figures showing that mammalian miRNAs have an effect of suppressing the induction of LPS-stimulated expressions of inflammatory cytokines in experiments in vitro using cultured cells, in an example of the present invention.

### DETAILED DESCRIPTION

Embodiments of the present invention that was completed based on the aforementioned findings are described in detail referring to examples.

Unless otherwise noted in embodiments and examples, all procedures used are according to standard protocols such as M. R. Green & J. Sambrook (Ed.), Molecular cloning, a laboratory manual (4th edition), Cold Spring Harbor Press, Cold Spring Harbor, New York (2012): F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd., with or without modifications or changes. In addition, if commercial reagent kits or measurement instruments are used, protocols attached thereto are used unless otherwise noted.

Objects, characteristics, advantages, and ideas of the present invention are apparent to a person skilled in the art from the description of the present specification and the person skilled in the art can easily reproduce the present invention from the description of the present specification. Embodiments of the present invention, specific examples thereof and so forth, which are described in the following, are described for exemplification or explanation, and the present invention is not limited thereto. It is apparent to the person skilled in the art that various alterations and modifications can be made on the basis of the description of the present specification within the spirit and the scope of the present invention disclosed in the present specification.

### Method for Preparing Extracellular Vesicles

Methods for preparing extracellular vesicles (also denoted as “EVs” in the present specification) derived from a plant are known to those skilled in the art and any of the methods can be used. Here, the plant is preferably selected from the group consisting of coastal hog fennel, Madeira vine, lemongrass, green garlic, turmeric, crimson glory vine, and bitter melon, but any plant can be used. Any portion of the plants can be used, and a person skilled in the art can choose an appropriate portion. It is, however, preferable to use leaves for coastal hog fennel, Madeira vine, lemongrass, and green garlic, rootstocks for turmeric, and fruits for crimson glory vine and bitter melon. Now, exemplified methods for preparing extracellular vesicles are described specifically.

First, plants are crushed to obtain juice. In this process, it is preferable to remove visible solids from the juice by centrifuging the juice at, for example, 1,000-10,000 g for 10-60 minutes. The juice is then ultracentrifuged at 120,000-150,000 g for 60 minutes to overnight to obtain precipitates. The precipitates are suspended in a buffer such as PBS, and a gradient is fractionated using a method such as the sucrose density gradient ultracentrifugation. For the sucrose density gradient, the target extracellular vesicles can be obtained by collecting fractions: fractions of 30-45% sucrose for coastal hog fennel; bands in fractions of 8-30% sucrose (corresponding to densities of 1.02-1.12 g/mL) (first band) and 30-45% sucrose (densities of 1.12-1.18 g/mL) (second band) for Madeira vine: fractions of 30-45% sucrose (densities of 1.12-1.18 g/mL) (second band) for lemongrass; fractions of 8-30% sucrose (densities of 1.02-1.12 g/mL) (first band) and 30-45% sucrose (densities of 1.12-1.18 g/mL) (second band) for green garlic: 30-45% sucrose (densities of 1.12-1.18 g/mL) (second band) for turmeric: 30-45% sucrose (densities of 1.12-1.18 g/mL) (second band) for crimson glory vine; and 30-45% sucrose (densities of 1.12-1.18 g/mL) (second band) for bitter melon. This preparation method can be appropriately chosen; for example, OptiPrep density gradient centrifugation, size exclusion chromatography, commercially available spin columns such as exoEasy midi spin columns, or tangential flow filtration can be used in place of the sucrose density gradient ultracentrifugation. Particle diameters, particle size distributions, and concentrations of the extracellular vesicles obtained can be measured using a nanoparticle analyzer such as NanoSight NS300 (Malvern Instrument).

An average particle diameter of the extracellular vesicles derived from coastal hog fennel is preferably in the range of 125-155 nm, and more preferably 135-145 nm. An average particle diameter of the extracellular vesicles corresponding to the first band for Madeira vine is preferably in the range of 125-155 nm, and more preferably 135-145 nm. An average particle diameter of the extracellular vesicles corresponding to the second band for Madeira vine is preferably in the range of 135-165 nm, and more preferably 145-155 nm. An average particle diameter of the extracellular vesicles derived from lemongrass is preferably in the range of 135-165 nm, and more preferably 145-155 nm. An average particle diameter of the extracellular vesicles corresponding to the first band for green garlic is preferably in the range of 125-155 nm, and more preferably 135-145 nm. An average particle diameter of the extracellular vesicles corresponding to the second band for green garlic is preferably in the range of 135-165 nm, and more preferably 145-155 nm. An average particle diameter of the extracellular vesicles derived from turmeric is preferably in the range of 110-140 nm, and more preferably 120-130 nm. An average particle diameter of the extracellular vesicles derived from crimson glory vine is preferably in the range of 160-190 nm, and more preferably 170-180 nm. An average particle diameter of the extracellular vesicles derived from bitter melon is preferably in the range of 125-155 nm, and more preferably 135-145 nm. For the particle diameter of each extracellular vesicle derived from a plant, 80% or more, preferably 90% or more, and more preferably, 95% or more of the extracellular vesicles have a particle diameter in the range of 50-250 nm.

Coastal hog fennel is a plant belonging to the Peucedanum genus in the Apiaceae family, with the scientific name Peucedanum japonicum. Madeira vine, also known as mignonette vine or lamb's tail, is a plant belonging to the Anredera genus in the Basellaceae family, with the scientific name Anredera cordifolia. Lemongrass is a plant belonging to the Cymbopogon genus in the Poaceae family, with the scientific name Cymbopogon citratus. Green garlic refers to garlic (scientific name: Allium sativum) with green shoots on the head grown from December to February. Turmeric, also known as Indian saffron, is a plant belonging to Curcuma genus in the Zingiberaceae family, with the scientific name Curcuma longa. Crimson glory vine is a plant belonging to the Vitis genus in the Vitaceae family, with the scientific name Vitis coignetiae. Bitter melon, also known as bitter gourd or balsam-pear, is a plant belonging to the Momordica genus in the Cucurbitaceae family, with the scientific name Momordica charantia var. pavel.

### Expression Suppressors Containing Extracellular Vesicles

Suppressors of expression of inflammatory cytokines disclosed in the present specification contain extracellular vesicles derived from a plant as an active ingredient. The inflammatory cytokines includes IL-6, IL-1β, CXCL-2, IL-4, IL-13, IL-33, and TNF-α.

Suppressors of expression of senescence markers disclosed in the present specification contain extracellular vesicles derived from a plant as an active ingredient. The senescence markers include p15, p16, p19, and β-galactosidase. Thus, the extracellular vesicles derived from a plant have an anti-senescence effect.

### Medicaments Containing Extracellular Vesicles

Pharmaceutical compositions and products disclosed in the present specification contain extracellular vesicles derived from a plant as an active ingredient.

Diseases or conditions to be treated by the pharmaceutical products include inflammation, diabetes, obesity, dry eye syndrome, irritable bowel syndrome, cytokine storm, and dementia. Thus, the pharmaceutical products can be used as anti-inflammatory agents, muscle strength enhancers, therapeutic agents for diabetes, prophylactic agents for diabetes, agents for reducing subcutaneous fat, slimming agents, therapeutic agents for dry eye syndrome, therapeutic agents for irritable bowel syndrome, cytokine storm inhibitors, therapeutic agents for coronavirus infection, or therapeutic agents for dementia. The inflammation to be treated by the anti-inflammatory agents include encephalitis, myalgic encephalomyelitis, chronic fatigue syndrome, post-coronavirus diseases, inflammatory bowel diseases or dermatitis.

The pharmaceutical products are preferably in the form of an ointment, an oral medicament, eye drops, or an injection, depending on their applications. For injections, any route of administration can be used, but intravenous administration is preferable.

Method for Producing Small RNAs Derived from Extracellular Vesicles

Small RNAs (including miRNAs herein) derived from extracellular vesicles described in the present specification comprise or consist of one or some of the following sequences. In the following listing, sequences of small RNAs in the extracellular vesicles derived from coastal hog fennel which correspond to those of mammalian miRNA are denoted as “Pj-(name of miRNA corresponding to its mammalian version).” The bases denoted in lowercase letters in a plant small RNA indicate that they differ from bases of its corresponding mammalian miRNA.

hsa-miR-6737-3p:
(SEQ ID NO: 1)
UCUGUGCUUCACCCCUACCCAG
mmu-miR-7006-3p:
(SEQ ID NO: 2)
UUUCUGACCUGGAUCCCCAG
mmu-miR-6995-3p:
(SEQ ID NO: 3)
UGUGUCCCCUUCCUCUCACAG
mmu-miR-12178-5p:
(SEQ ID NO: 4)
UAUGGGUGGCUGGUCUCAGAAGAG
mmu-miR-3063-3p:
(SEQ ID NO: 5)
UGAGGAAUCCUGAUCUCUCGCC
mmu-miR-487b-5p:
(SEQ ID NO: 6)
UGGUUAUCCCUGUCCUCUUCG
Pj-has-miR-6737-3p:
(SEQ ID NO: 23)
UCUG(a/c)GCU(a/g)CACCCCUACCCAG
Pj-mmu-miR-7006-3p:
(SEQ ID NO: 24)
UUUCUGAgCUGcAUCCCCAG
Pj-mmu-miR-6995-3p:
(SEQ ID NO: 25)
UGUGUCCCCaUCgUCUCACAG
Pj-mmu-miR-12178-5p:
(SEQ ID NO: 26)
UAaGGGUGGCUGtUCUCAGAAGAG
Pj-mmu-miR-3063-3p:
(SEQ ID NO: 27)
aGgGGAAUCCUGAUCUCUCGCC
Pj-mmu-miR-487b-5p:
(SEQ ID NO: 28)
UGGUUAUCCCUccCCUCUUCG

These small RNAs can be produced by chemical synthesis known to those skilled in the art.

As long as effects of the present invention can be obtained, 1, 2, 3, 4, or 5 bases of the small RNA sequences may be mutated to other base(s), and at least one and up to all (e.g., 1, 2, 3, 4, 5, 6, 7, 8, 9, 10, 11, 12, 13, 14, 15, 16, 17, 18, 19, or 20) bases may be DNAs or modified bases. Moreover, the small RNAs may contain 1, 2, 3, 4, or 5 extra sequence(s) of RNA or DNA.

Expression Suppressors Containing Extracellular Vesicles and/or Small RNAs Derived Therefrom

The suppressors of expression of inflammatory cytokines disclosed in the present specification contain one or more extracellular vesicles of plant origin and/or small RNAs derived therefrom as ingredients. The inflammatory cytokines includes, for example, IL-6, IL-1β, CXCL2, IL-4, IL-13, IL-33, and TNF-α. Thus, the extracellular vesicles of plant origin and/or small RNAs derived therefrom have an anti-inflammatory effect.

The suppressors of expression of senescence markers disclosed in the present specification contain extracellular vesicles of plant origin and/or small RNAs derived therefrom as active ingredients. The senescence markers include p15, p16, p19, and β-galactosidase. Thus, extracellular vesicles of plant origin and/or small RNAs derived therefrom have an anti-senescence effect.

Medicaments Containing Extracellular Vesicles and/or Small RNAs Derived Therefrom

The pharmaceutical compositions and products disclosed in the present specification contain one or more extracellular vesicles of plant origin and/or small RNAs derived therefrom as active ingredients.

Diseases or conditions to be treated by the pharmaceutical products include inflammation, diabetes, obesity, dry eye syndrome, irritable bowel syndrome, cytokine storm, and dementia. Thus, the pharmaceutical products can be used as anti-inflammatory agents, muscle strength enhancers, therapeutic agents for diabetes, prophylactic agents for diabetes, agents for reducing subcutaneous fat, slimming agents, therapeutic agents for dry eye syndrome, therapeutic agents for irritable bowel syndrome, cytokine storm inhibitors, therapeutic agents for coronavirus infection, or therapeutic agents for dementia. The inflammation to be treated by the anti-inflammatory agents include encephalitis, myalgic encephalomyelitis, chronic fatigue syndrome, post-coronavirus diseases, inflammatory bowel diseases or dermatitis.

The pharmaceutical products are preferably in the form of an ointment, an oral medicament, eye drops, or an injection, depending on their applications. For injections, any route of administration can be used, but intravenous administration is preferable.

Food Products Containing Extracellular Vesicles and/or Small RNAs Derived Therefrom

The food compositions and products disclosed in the present specification contain extracellular vesicles of plant origin and/or small RNAs derived therefrom.

The food compositions may be used to produce food products consumed regularly on a daily basis, or food products containing an ingredient with function such as health foods, functional foods, foods for specified health uses, and supplements. The food products can be in any of various forms such as solids, liquids, jellies, gummies, gums, and syrups.

Food products containing extracellular vesicles of plant origin and/or small RNAs derived therefrom have a function of preventing or treating inflammation, diabetes, obesity, irritable bowel syndrome, cytokine storm, and dementia. Thus, the food products can be used for the purposes of preventing inflammation, strengthening muscle, treating diabetes, preventing diabetes, suppressing subcutaneous fat, reducing body weight, treating irritable bowel syndrome, inhibiting cytokine storm, treating coronavirus infection, or treating dementia. As supplements, they can be used as anti-inflammatory agents, muscle strength enhancers, therapeutic agents for diabetes, prophylactic agents for diabetes, agents for reducing subcutaneous fat, slimming agents, therapeutic agents for irritable bowel syndrome, cytokine storm inhibitors, therapeutic agents for coronavirus infection, or therapeutic agents for dementia. The inflammation to be alleviated by the anti-inflammatory effect include encephalitis, myalgic encephalomyelitis, chronic fatigue syndrome, post-coronavirus diseases, inflammatory bowel diseases or dermatitis.

The food products are taken orally.

Cosmetic Products Containing Extracellular Vesicles and/or Small RNAs Derived Therefrom

The cosmetic compositions and products disclosed in present specification contain extracellular vesicles of plant origin and/or small RNAs derived therefrom.

The cosmetic products containing extracellular vesicles of plant origin and/or small RNAs derived therefrom have an effect of preventing or treating inflammation, especially dermatitis. Thus, the cosmetic products can be used for anti-inflammation purposes.

The cosmetic products are applied on the skin as ointments.

### Examples

### (1) Preparation of Extracellular Vesicles

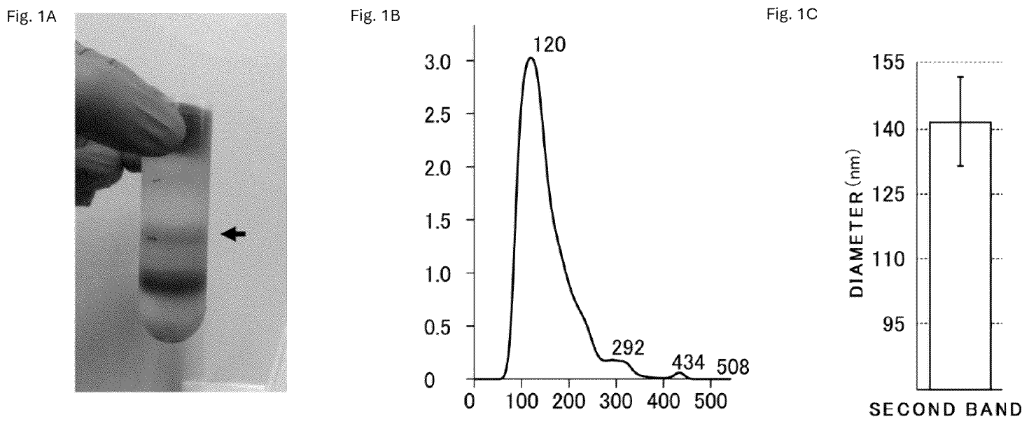

Coastal hog fennel plants cultivated at a farm on Miyako Island, Okinawa were washed with water and crushed using a juicer for home use (Kuvings) after having had water removed. The crushed plant thus obtained was subjected to centrifugation at 3,000 g for 20 minutes by using Avati J-E (Bechman Courlter: JA-14 rotor) and then at 10,000 g for 40 minutes to remove somewhat larger solids to obtain juice. The resulting juice was subjected to centrifugation at 150,000 g for 90 minutes using Optima XE-90 (Bechman Courlter: SW32 Ti rotor) to obtain precipitates. The precipitates were suspended in PBS, applied to a sucrose density gradient (8%, 30%, 45%, and 60%,) and further ultracentrifuged at 150,000 g for 90 minutes for the fractionation (FIG. 1A). Then, a band at densities 1.12-1.18 g/mL (an arrow in FIG. 1A) was collected using a 20-mL syringe (TERUMO CORPORATION). The collected fraction was mixed with PBS and subjected to centrifugation at 150,000 g for 90 minutes. The precipitates were suspended in PBS again as extracellular vesicles (Evs).

A particle size distribution (FIG. 1B) and an average particle diameter (FIG. 1C) of the extracellular vesicles thus obtained were measured using NanoSight NS300 (Malvern Instrument) with blue laser at 405 nm. As a result, 95% or more particles had a diameter in the range of 50-250 nm, and their average particle diameter was about 140 nm.

### (2) Inhibition of the Expression of Inflammatory Cytokines

In cell cultures in the Examples, minimum essential medium (MEM) powder and Dulbecco's modified eagle's medium (DMEM) powder were purchased from Nissui Pharmaceutical Co., Ltd. Fetal bovine serum (FBS) was purchased from Equitech-Bio or GIBCO. Trypsin powder was purchased from GIBCO. For other reagents not otherwise specified, special-grade reagents for biochemical experiments available from Wako, Nacalai Tesque, Inc., and SIGMA were used.

Mouse macrophage Raw-Blue cells (InvivoGen) and human macrophage THP-1 cells (ATCC) were used after seeded at 4.7×10⁶ cells in 100-mm dishes using Dulbecco's modifeid eagle medium (DMEM: Thermo Fisher) containing 10% FBS, 100 μg/mL Normocin™ (InvivoGen) and 100 U/mL PS (Pen-Strep) and cultured over several passages at 37° C. in 5% CO₂.

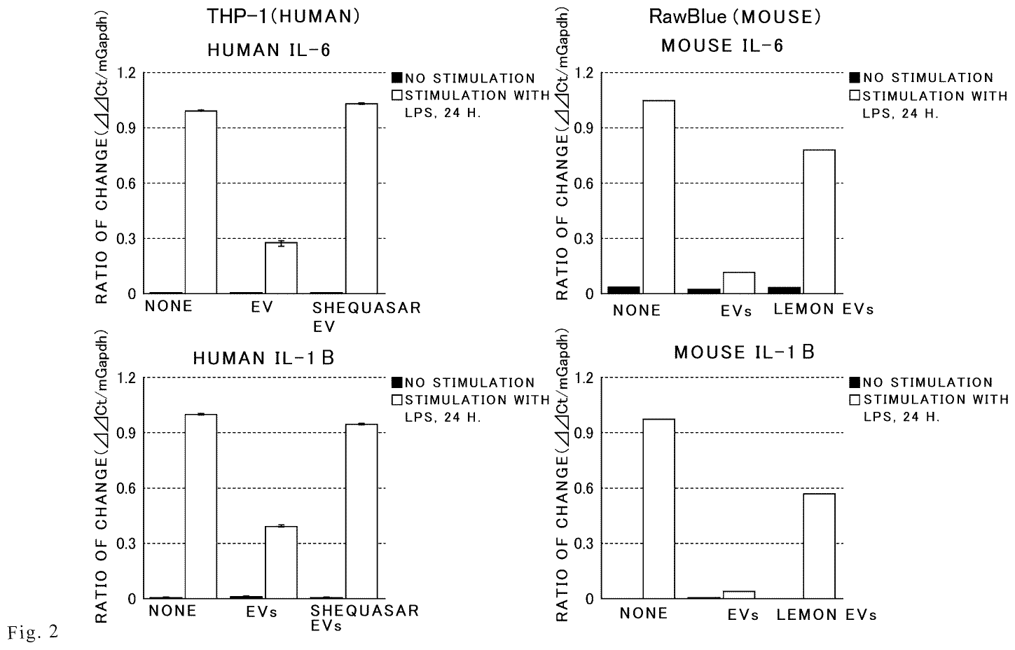

First, Raw-Blue or THP-1 cells were seeded onto 12-well dishes at 0.8×10⁵ cells/well, and extracellular vesicles were added thereto at 1.0×10¹⁰ vesicles/mL. After 16 hours, the cells were washed with PBS, and LPS (of Salmonella minnesota R595: WAKO) (final concentration of 1 g/mL) was added to the culture medium to stimulate the cells for 24 hours. After the stimulation, the cells were washed with PBS, and total RNAs were extracted using Trizol (Thermo Fisher). Then, the expressions of inflammatory cytokines IL-6, IL-33, IL-1β, and TNF-α were measured using RT-qPCR. The results are shown in FIG. 2.

As shown in FIG. 2, in both cell lines, the extracellular vesicles derived from coastal hog fennel strongly suppressed the LPS-induced expression of IL-6 and IL-1β.

### (3) Anti-Inflammatory Effects in Mouse Models of Colitis

10¹⁰ extracellular vesicles were administered to 8-week-old wild-type male mice (C57BL6/6J) once a day for 1 week. Then, 3% sodium dextran sulfate (DSS) that induces colitis was added to their drinking water, after which the same amounts of extracellular vesicles were kept administered.

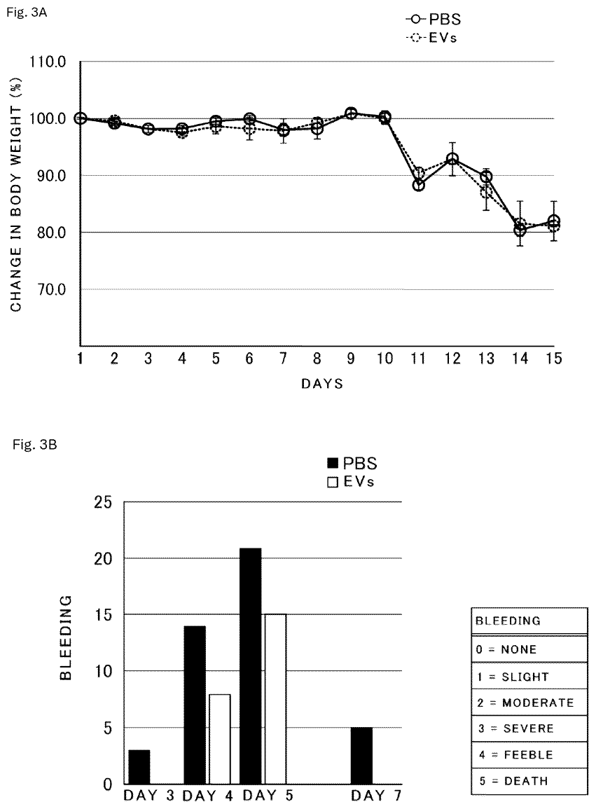

As shown in FIG. 3A, the mouse group to which the extracellular vesicles were administered without the DSS treatment exhibited similar weight gain to those in the control group administered with PBS, indicating that the extracellular vesicles had no acute toxicity to the animals.

Moreover, as shown in FIG. 3B, blood was found in stool on and after Day 3 of the DSS intake in the PBS-administered group, and fecal blood increased as days after administration passed by, while in the EV-administered group no blood was found in stool on Day 3: blood was found in stool but at a lower level after Day 4; and blood was no longer found on Day 7.

Next, after the administration of DSS to the mouse group with the intake of PBS and the mouse group with the intake of extracellular vesicles, they were fixed by perfusion on Days 0, 3, 5, and 7. Their large intestines from the appendix to the anus were rolled up (using the Swiss roll technique) to prepare paraffin-embedded sections. The sections were stained with HE, and then with Alcian Blue (pH 2.5). Subsequently, cell nuclei were stained red with Nuclear Fast red and then colorectal sections were observed.

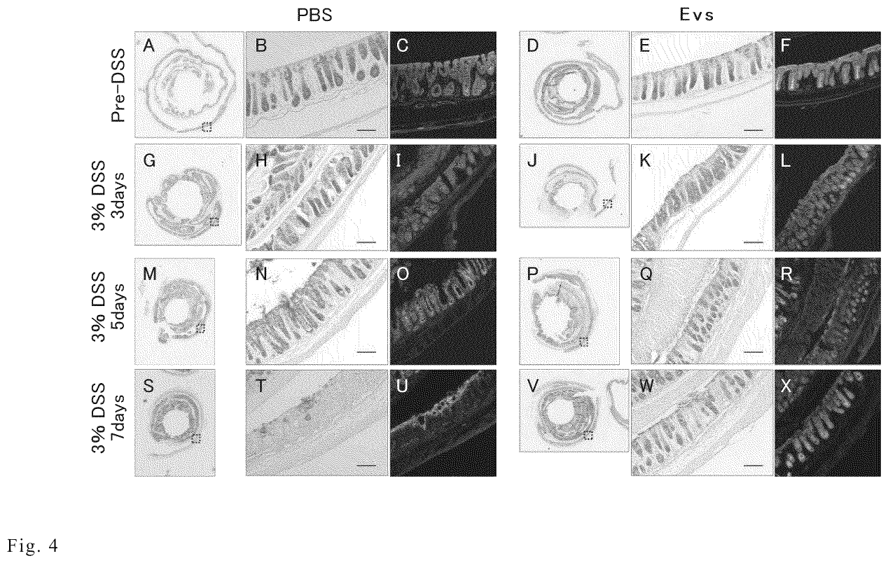

As shown in FIG. 4, for proximal, intermediate, and distal colorectal regions defined in order of proximity to the appendix, almost the entire colorectal regions were stained with Alcian Blue that binds to carboxylate and sulfate groups of acid mucopolysaccharides in both PBS-administered group and EV-administered group (A, B, D, and E in FIG. 4). Moreover, immunostaining using β-catenin and ZO-1, both of which are markers of epithelial cells, indicated that the regions stained with Alcian Blue were epithelial cells (A, B, D, and E in FIG. 4).

On Day 3 of the DSS intake, no significant tissue damage was observed in the images with either Alcian Blue staining or immunostaining (G, H, J, and K in FIG. 4). On Day 5 of the DSS intake, however, Alcian Blue leaked out of the cells in the tissue of the PBS intake group (M and N in FIG. 4), and punctuations were found in the regions stained with β-catenin due to the change in tight-junction associated with the reduced expression of ZO-1 (O in FIG. 4). In contrast, no significant changes were observed on Day 5 of the DSS intake in the EV-administered group (P, Q, and R in FIG. 4). Moreover, on Day 7 of the DSS intake, the cells positive for Alcian Blue were almost disappeared in the distal colorectal region of the PBS intake group, and simple columnar epithelium structures were significantly damaged (S, T, and U in FIG. 4), whereas no significant leakage of Alcian Blue out of the cells was found with the epithelial structure being maintained, although the decrease in expression of ZO-1 was observed in the distal colorectal region in the EV-administered group (V, W, and X in FIG. 4).

Thus the extracellular vesicles have an anti-inflammatory effect in colitis.

### (4) Suppression of the Induction of Expression of Senescence Markers

Effects of extracellular vesicles on cellular senescence and its associating inflammatory responses were examined using a cultured cell system that causes cellular senescence by inducing the expression of Ras gene in a drug-dependent manner in normal human fibroblast cells IMR90

The IMR90 cells were used after seeding the cells at 1×10⁶ cells in 10-cm dishes and culturing them over several passages in MEM medium supplemented with 10% FBS, 1×non-essential amino acids solution, and 1 mM sodium pyruvate in a hypoxic incubator at 37° C. in 3% O₂ and 5% CO₂. The Plat A cells were used after seeding them at 1×10⁶ cells in 10-cm dishes and cultured over several passages in DMEM supplemented with 10% FBS in an incubator at 37° C. in 5% CO₂.

First, Plat A cells were transfected with the retroviral vector pLNCX2 ER: Ras (Addgene) using PEI to perform the packaging of ER: Ras-expressing retrovirus. The culture medium was replaced after 12 hours of the transfection, and the culture supernatant containing the retrovirus was collected 24-36 hours later than the replacement. Two days after the retrovirus infection of IMR90 cells, the infected cells, IMR90 ER-Ras were selected by passaging the cells in medium containing neomycin.

Cellular senescence by Ras induction was induced by treating IMR90 ER-Ras cells with 200 nM 4-OH tamoxifen for 6 days. 1×10¹⁰ vesicles/mL of extracellular vesicles were added to the culture medium of the cells thus induced to senescence. Cells were harvested on Day 6, and RNA was extracted using TRIsure (Nippon Genetics). cDNA was synthesized using Primescript RT Master Mix (Takara). The expressions of cellular senescence markers p15 and p16 and the inflammatory cytokine IL-6 were examined by RT-qPCR. As controls, cells without the Ras induction and cells without the addition of extracellular vesicles after the Ras induction were also treated in the same way.

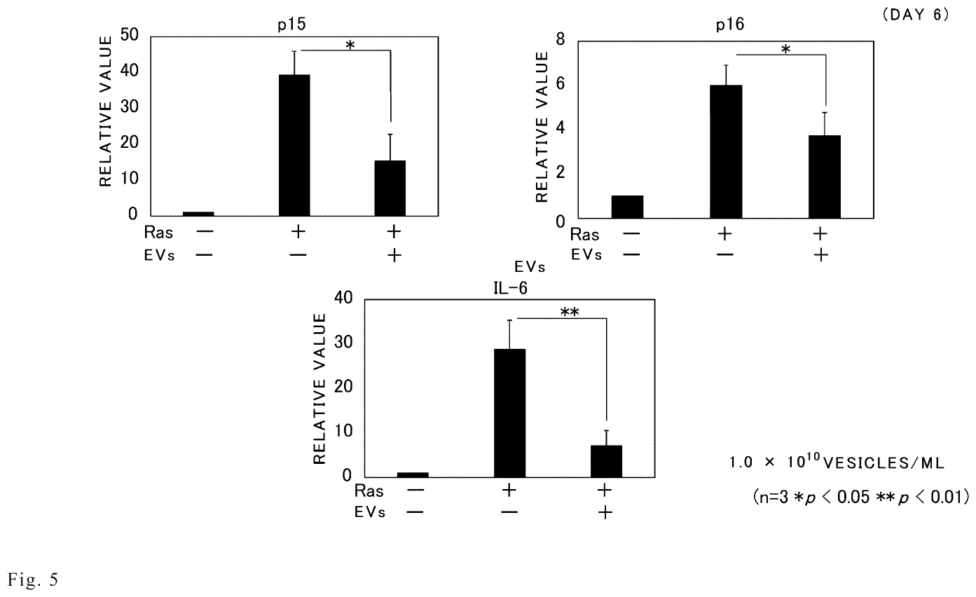

As a result shown in FIG. 5, the induction of expressions of cellular senescence markers p15 and p16 and inflammatory cytokine IL-6 associated with the Ras induction were suppressed by adding the extracellular vesicles. Similar effects were observed in the experimental system in which cellular senescence was induced by Sodium butyrate: NaBu in place of the Ras induction.

Thus, the extracellular vesicles derived from coastal hog fennel have a function of suppressing cellular senescence upon Ras induction.

### (5) Effect of Enhancing Muscle Strength in Mice

To quantitatively evaluate an effect of extracellular vesicles derived from coastal hog fennel on the muscle strength of mice, the Grip Strength Test and the 4-limb Hanging Test were performed to measure muscle strength (especially grip strength) in high-fat-fed mice that were individually housed on a high-fat diet and to which the extracellular vesicles derived from coastal hog fennel had been administered. Specifically, after acclimation, 5-week-old male C57B6J mice were divided equally into two groups, with 9-10 animals in each group according to their body weight, and each mouse was fed a high-fat diet (60 kcal % Fat) for 12 weeks to create high-fat-fed mice. Extracellular vesicles derived from coastal hog fennel were added to pure water at a concentration of 3×10⁹ vesicles/mL and supplied to the mice through their drinking water.

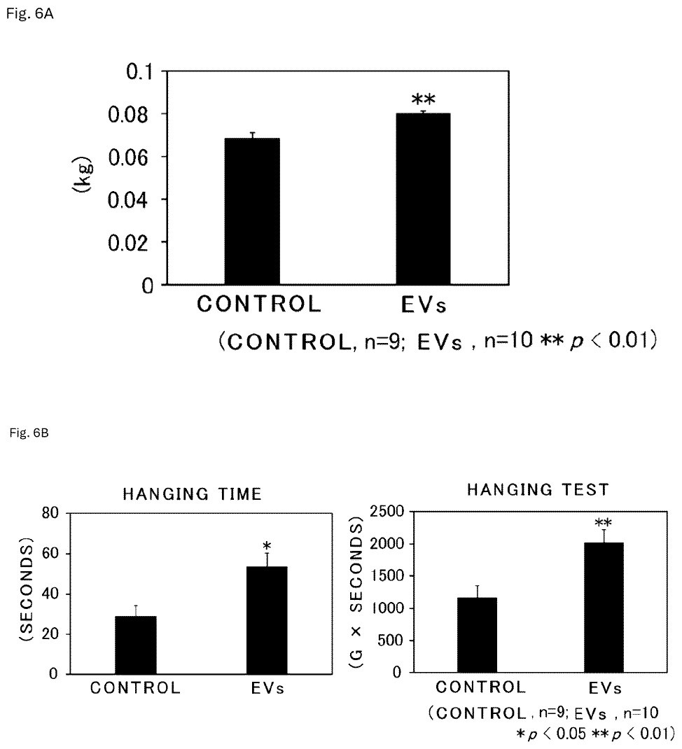

For the Grip Strength Test, the high-fat-fed mice were tested with Smart Grip Strength Meter for Rats and Mice MK-380Si (Muromachi Machinery Co., Ltd.), three times per mouse, and an average value was used as a grip strength of each mouse (FIG. 6A). For the 4-limb Hanging Test, with a mouse placed on a mesh grid gripping the grid with all its limbs, the grid was quickly flipped upside down. The length of time that the mouse was able to hang from the grid before falling is referred to as a “hanging time,” and the hanging time was multiplied by the body weight of the corresponding mouse to calculate a “hanging score.” The muscle strengths of the mice were assessed according to their hanging scores (FIG. 6B). A Student's t-test was used to test for significant differences.

As a result shown in FIG. 6A, the mice in the coastal hog fennel-derived EV-administered group exhibited higher grip strengths than those in the control group. In addition, as shown in FIG. 6B, the mice in the coastal hog fennel-derived EV-administered group exhibited longer durations of time (hanging times) during which the mice were able to hang from the grid in an upside-down position than those in the control group. The mice also exhibited larger scores (hanging scores) obtained by multiplying the respective hanging times by the body weight of the corresponding mice. This means that these mice have a higher muscular endurance.

Thus, the extracellular vesicles derived from coastal hog fennel have an effect of enhancing muscle strengths.

### (6) Anti-Diabetic Effects in Mice

High-fat-fed mice prepared in the same way as the above (5) were fasted for 4 hours, and then an insulin tolerance test was performed by intraperitoneally injecting human recombinant insulin (Invitrogen) diluted in PBS to 1 IU (International Unit)/kg into mice. Blood was collected from the tail vein at 0, 40, 80, and 120 minutes after the insulin injection, and blood glucose levels were measured using Ascensia Breeze 2 (Bayer Yakuhin, Ltd.). A Student's t-test was used to test for significance differences.

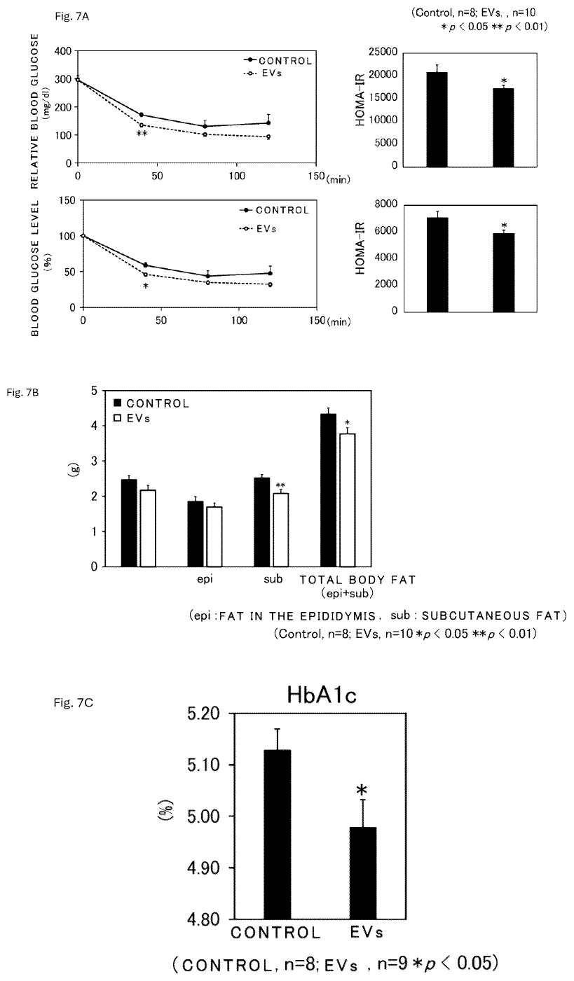

As a result shown in FIG. 7A, insulin resistance on a high-fat diet was reduced by the administration of the extracellular vesicles derived from coastal hog fennel.

Next, mice to which extracellular vesicles had been administered on the aforementioned high-fat diet were fed and watered for 12 weeks, fasted for 6 hours, and then slaughtered. Weights of their liver fat, fat in the epididymis, and subcutaneous fat were measured.

As a result shown in FIG. 7B, the weights of the fat tended to totally decrease by administering the extracellular vesicles derived from coastal hog fennel, and in particular, significant reductions were found in the weights of their subcutaneous fat.

Moreover, as described above, triglyceride, total cholesterol, LDL cholesterol, glucose, and HbA1c in blood samples collected from the high-fat-fed mice were quantified. As a result shown in FIG. 7C, the values tended to totally decrease by administering the extracellular vesicles derived from coastal hog fennel, and in particular, HbA1c values were significantly reduced.

Thus, the extracellular vesicles derived from coastal hog fennel have anti-diabetic and slimming effects.

### (7) Effects of Alleviating Dry Eye Syndrome

Symptoms of dry eye syndrome include decrease in tear volume and damages to corneal epithelial cells. In this Example, mouse models in which symptoms common to those of human dry eye syndrome can be induced (Ouyang W. et al., Invest Ophthalmol Vis Sci. 2021 January; 62 (1): 25.; Lin Z. et al., Mol Vis. 2011:17: 257-264) were used to show that extracellular vesicles derived from coastal hog fennel are effective in reducing the symptoms of the dry eye syndrome.

Specifically, dry eye syndrome was induced in 8- to 10-week-old wild-type C57BL/6J mice by ophthalmic injection of a 0.1% BAC solution. The 0.1% BAC solution was prepared by diluting BAC purchased from Sigma to 0.1% using saline solution. The extracellular vesicles derived from coastal hog fennel to be dropped were diluted in saline to a concentration of 2.5×10¹⁰ vesicles/mL, and 5 μl of the solution was applied to each eye of the mice twice a day. For the first five days, the animals in the EV-applied group received the eye drops of extracellular vesicles in their eyes, whereas those in the control group received eye drops of saline solution. For the following 7 days, 0.1% BAC solution was applied to induce corneal damage, one of the symptoms of dry eye syndrome. After 30 minutes, the animals in the control group received eye drops of saline solution, whereas those in the EV-applied group received eye drops of extracellular vesicles in their eyes. For the last 7 days, the animals in the control group received eye drops of saline solution, whereas those in the EV-applied group received eye drops of extracellular vesicles in their eyes.

After 19 days of treatment, the tear volumes of the EV-applied group and the control group were measured as follows. A cotton thread (ZONE-QUICK/Ayumi Pharmaceutical Co., Ltd.) was inserted into the lower eyelid and removed after 20 seconds. The length of a segment of the thread with its color changed by being wetted by tears was measured under a stereomicroscope as tear volume. A Student's t-test was used to test for significant differences in tear volume.

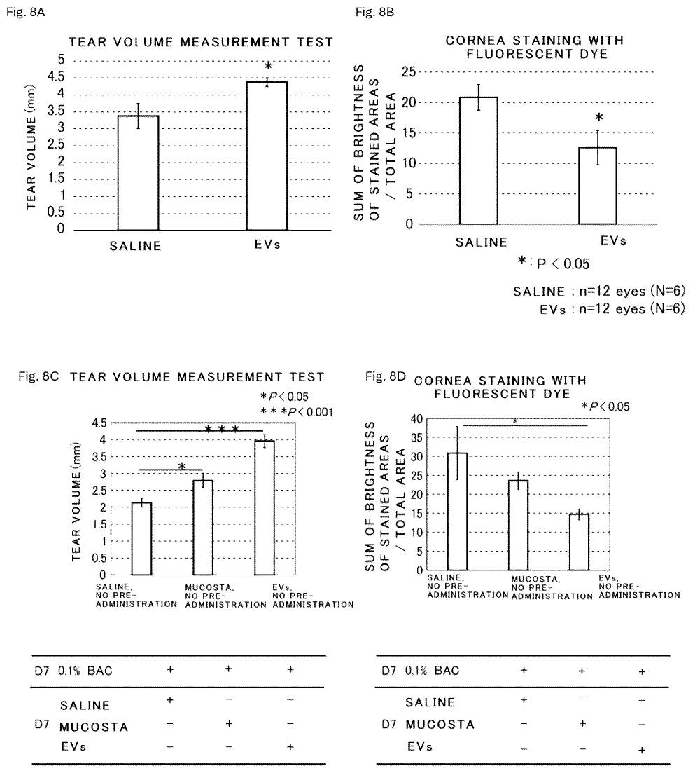

As a result shown in FIG. 8A, the tear volume was significantly higher in the EV-applied group than in the control group (the control: 3.38±0.37 vs. the EV-applied group: 4.38±0.13 mm).

Next, corneal damage caused by the 0.1% BAC solution was examined by staining the cornea with a fluorescent dye. After 19 days of treatment, 1 μl of 0.1% fluorescein sodium salt solution was applied to each eye of the mice in all groups, and images of the corneal surface were acquired with a digital camera (Lumix) connected to a fluorescent stereomicroscope (Leica). The healing of the cornea after inflammation was assessed based on the sum of the brightness of the areas stained with the fluorescent dye relative to the total area of the corneal surface. Here, the area where the cornea is damaged is stained with the fluorescent dye.

As a result shown in FIG. 8B, the stained area for the EV-applied group was significantly decreased compared to that for the control group (the control: 20.86±2.09 vs. the EV-applied group: 12.58±2.8), indicating that the corneal damage had been healed by the application of the eye drops of the extracellular vesicles.

Furthermore, comparative experiments with Mucosta that is currently used in ophthalmology clinical practice, were conducted. In this Example, either Mucosta or a solution of extracellular vesicles was applied to the eyes of the animals after the 7-day applications of eye drops of 0.1% BAC. Tear volumes and recoveries from corneal damages were measured as described above.

As a result shown in FIGS. 8C and 8D, the extracellular vesicles had a larger effect on both tear volume and recovery from corneal damages than Mucosta.

Thus, the extracellular vesicles derived from coastal hog fennel have a therapeutic effect on dry eye syndrome.

### (8) Effects of Treating Dermatitis

The abdomen of 8-week-old male mice (Balb/c) was shaved and sensitized by applying 5% 2,4,6-trinitrochlorobenzene (TNCB: Tokyo Chemical Industry Co., Ltd.) dissolved in a mixed solvent of acetone and ethanol (1:9) (150 μl/animal) to the abdomen. On Days 3-4, extracellular vesicles derived from coastal hog fennel (1.0×10¹¹ vesicles/100 μl PBS/animal) was injected into the mice intraperitoneally. The control group received an intraperitoneal injection of PBS (100 μl/animal). On Day 5, 1% TNCB dissolved in acetone was applied to the front and back of both ears in an amount of 10 μl each to induce contact dermatitis. In the non-induced control group, acetone was applied instead of 1% TNCB dissolved in acetone. After 24 hours, the thickness of the auricle was measured with a micrometer. Measurements were taken in both ears, and an average value was used as the thickness of the auricle of the individual animals.

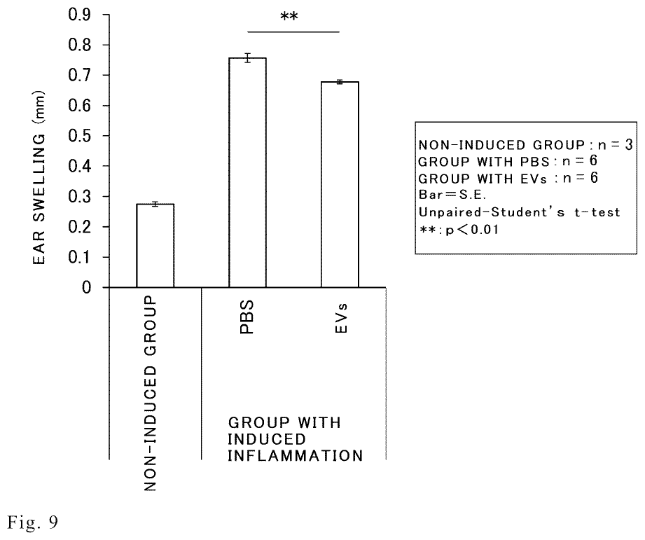

As a result shown in FIG. 9, the ear swelling associated with inflammation was significantly reduced in the EV-administered group compared to the control group (i.e., the PBS-administered group).

After the thicknesses were measured, cells for FACS analyses were collected from the right auricle, and RNAs for qPCR were collected from a portion of the left auricle. The remainder of the left auricle was fixed in 10% formalin/PBS and embedded in paraffin, and then tissue sections were prepared and stained with HE.

The right ear for FACS analyses was cut into small pieces after the front-side and back-side skins were peeled with tweezers. Next, they were dissociated into single cells using Multi tissue dissociation kit 1 (Miltenyi) and gentleMACS Octo Dissociator with Heaters (Miltenyi). The dissociated cells were passed through a 100-μl cell strainer and blood cells were lysed. Cells after lysis were suspended in MACS Rinsing buffer (Miltenyi), and the number of cells was counted. The resulting cells were blocked with anti-CD16/32 antibody and stained with anti-CD11b-FITC (M1/70) (a mononuclear cell marker), anti-F4/80-PE (BM8) (a macrophage marker), anti-Ly6G-PE-Cy7 (1A8) (a neutrophil marker), anti-CD3e-APC (145-2C11) (a T-cell marker), or anti-CD45/Cy7 (13/2.3) (a leukocyte marker) (all of the markers are available from BioLegend). Cells were stained with a cell viability solution (BD) and subjected to a FACS analysis using FACS Melody (BD) to examine the expression of inflammatory cytokines.

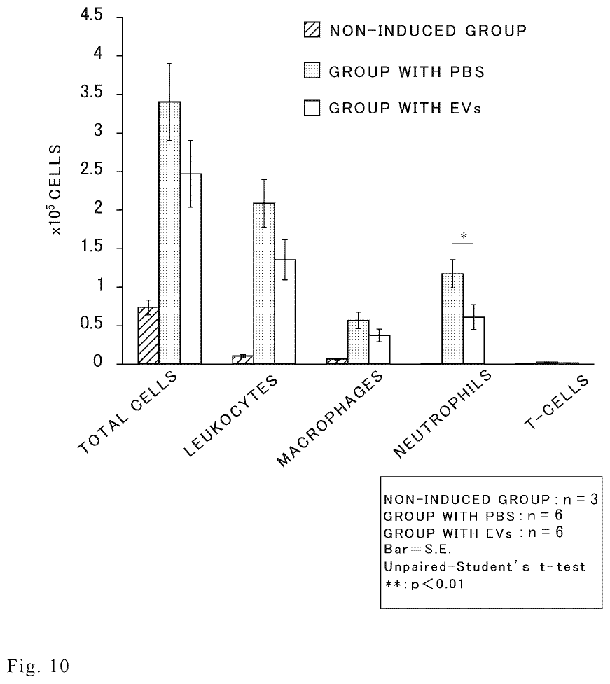

As a result shown in FIG. 10, the infiltration of immune cells associated with inflammation tended to be suppressed and, in particular, the infiltration of neutrophils was significantly suppressed in the EV-administered group. This result is consistent with the fact that the extracellular vesicles have an anti-inflammatory effect.

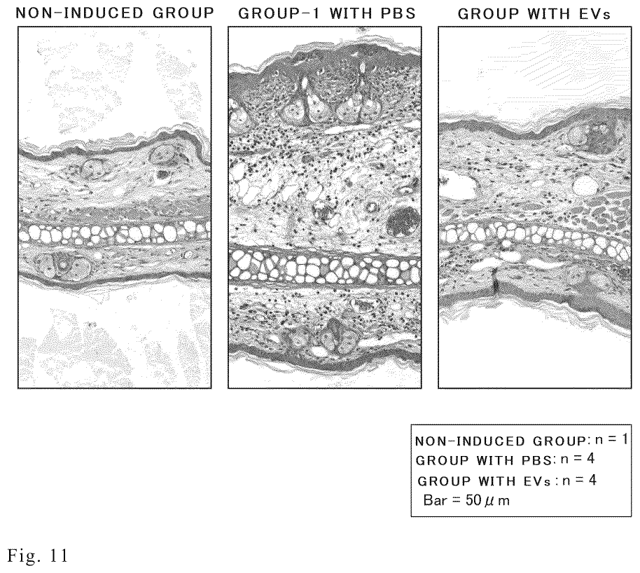

FIG. 11 shows the observed tissue sections. Swelling of ears associated with inflammation was found to be significantly suppressed in the EV-administered group compared to the control group (i.e., the PBS-administered group) in the data shown in FIG. 9. On the other hand, the results of the direct observation was consistent with the data in FIG. 9; that is, the auricles had the smallest and largest thicknesses in the non-induced control group and the PBS-administered group, respectively, and the thicknesses were decreased in the EV-administered group.

A portion of the left auricle was suspended in Trisure (Bioline) and subjected to homogenization. RNAs were collected according to the protocol attached to the reagent, and reverse transcription was performed using Primescript (TAKARA). qPCR was performed using SYBR Green qPCR Master Mix (Roche). In addition, PCR was performed with LightCycler 480 using the following primers to examine the expression of cytokines and chemokines involved in inflammation.

IL-4 for:
(SEQ ID NO: 7)
CATCGGCATTTTGAACGAG;
IL-4 rev:
(SEQ ID NO: 8)
CGAGCTCACTCTCTGTGGTG;
IL-13 for:
(SEQ ID NO: 9)
CCTCTGACCCTTAAGGAGCTTAT;
IL-13 rev:
(SEQ ID NO: 10)
CGTTGCACAGGGGAGTCT;
CXCL1 for:
(SEQ ID NO: 11)
AGACTCCAGCCACACTCCAA;
CXCL1 rev:
(SEQ ID NO: 12)
TGACAGCGCAGCTCATTG;
CXCL2 for:
(SEQ ID NO: 13)
AAAATCATCCAAAAGATACTGAACAA;
CXCL2 rev:
(SEQ ID NO: 14)
CTTTGGTTCTTCCGTTGAGG;
IL-33 for:
(SEQ ID NO: 15)
GACACATTGAGCATCCAAGG;
IL-33 rev:
(SEQ ID NO: 16)
AACAGATTGGTCATTGTATGTACTCAG;
IL-6 for:
(SEQ ID NO: 17)
GCTACCAAACTGGATATAATCAGGA;
IL-6 rev:
(SEQ ID NO: 18)
CCAGGTAGCTATGGTACTCCAGAA;
IL-1ß for:
(SEQ ID NO: 19)
AGTTGACGGACCCCAAAAG;
IL-1ß rev:
(SEQ ID NO: 20)
AGCTGGATGCTCTCATCAGG;
TNF-α for:
(SEQ ID NO: 21)
TCTTCTCATTCCTGCTTGTGG;
and
TNF-α rev:
(SEQ ID NO: 22)
GAGGCCATTTGGGAACTTCT.

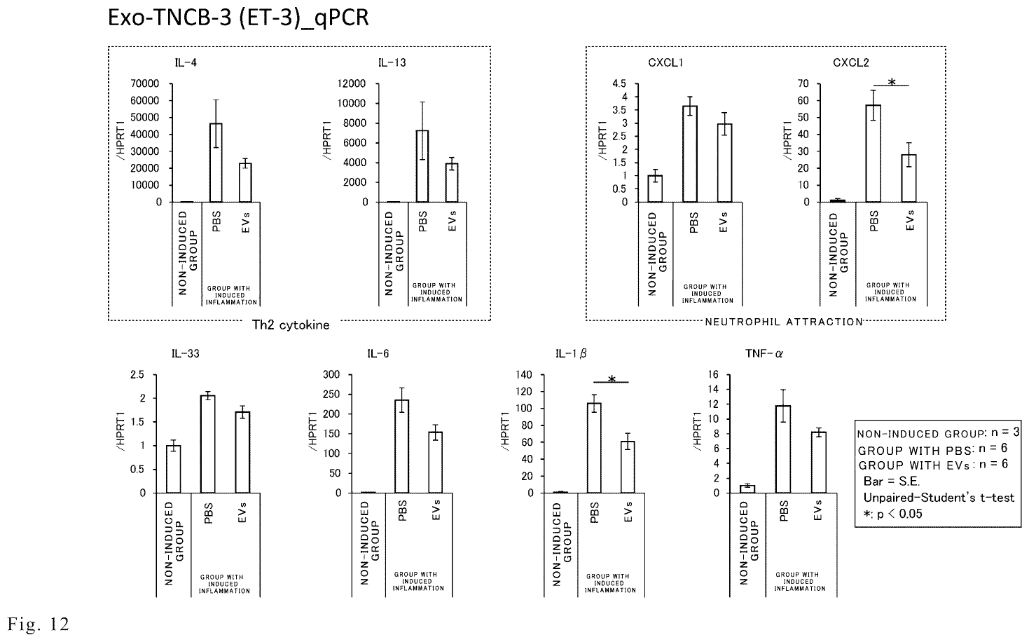

As a result shown in FIG. 12, the expression of inflammatory factors tended to be suppressed in the EV-administered group, among which the expressions of CXCL2 and IL-1ß were significantly decreased. CXCL2 has an activity to induce the migration and infiltration of neutrophils. It is considered that the lower expression of CXCL2 under the influence of the extracellular vesicles results in the suppression of the infiltration of neutrophils.

(9) Effects of Extracellular Vesicles Derived from Plants Other than the Coastal Hog Fennel on Suppressing the Induction of the Expression of Inflammatory Cytokines

In the same manner as the one described in (1) for the coastal hog fennel, extracellular vesicles were prepared from different types of plants (leaves of Madeira vine [137 and 145 nm], leaves of lemongrass [147 nm], leaves of green garlic [138 and 147 nm], rootstocks of turmeric [123 nm], fruits of crimson glory vine [176 nm], fruits of bitter melon [138 nm], leaves of aloe [150 and 187 nm], and fruits of Oriental melon [135 nm]) (the numerals in the square brackets denote average particle diameters of the extracellular vesicles derived from the respective plants]). It is noted that 95% or more particles were in the range of 50-250 nm in all samples.

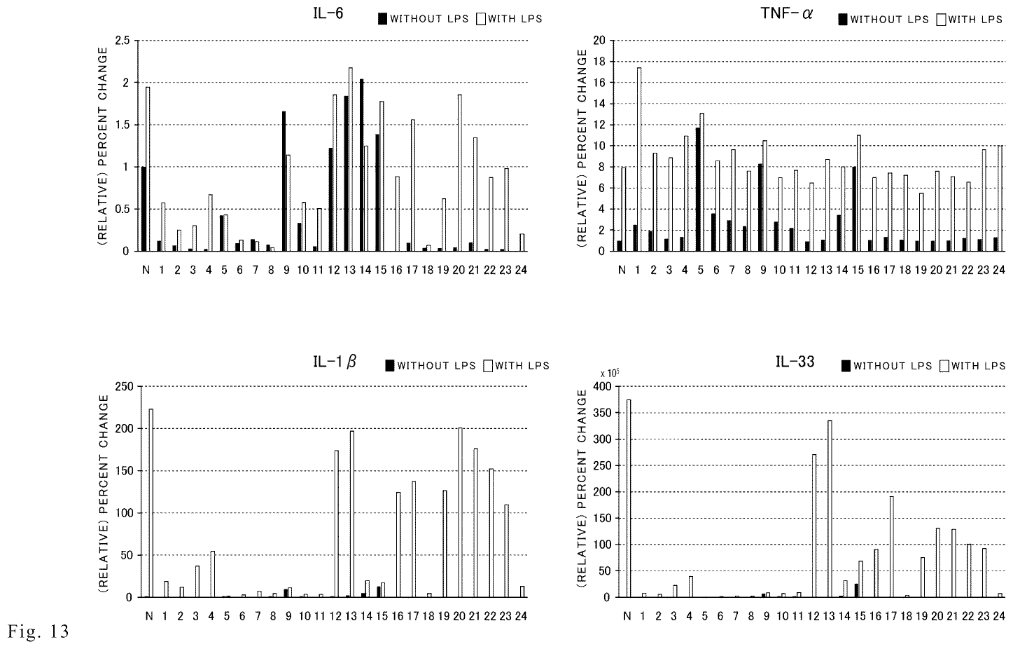

Raw-Blue cells, a mouse macrophage cell line, was exposed to the extracellular vesicles derived from each of the plants at a concentration of 1×10¹⁰ vesicles/mL for 16 hours and then stimulated with 1 μg/mL LPS (of Salmonella minnesota R595). After 24 hours, the same procedure as described in (8) was used to measure the expressions of inflammatory cytokines IL-6, IL-33, IL-1β, and TNF-α using RT-qPCR. The measurements were standardized using the expression of the Gapdh gene. In addition, relative values were calculated, setting the value of the control group without stimulation as 1. The results are shown in FIG. 13.

In FIG. 13, the numerals represent the following plants and the positions of the bands corresponding to the respective plants. N: the negative control (PBS); 1: a first band for coastal hog fennel (8-30%, densities 1.02-1.12 g/mL); 2: a second band for coastal hog fennel (30-45%, densities 1.12-1.18 g/mL); 3: a first band for Madeira vine (8-30%, densities of 1.02-1.12 g/mL); 4: a second band for Madeira vine (30-45%, densities 1.12-1.18 g/mL); 5: a first band for lemongrass (8-30%, densities 1.02-1.12 g/mL); 6: a second band for lemongrass (30-45%, densities 1.12-1.18 g/mL); 7: a first band for green garlic (8-30%, densities 1.02-1.12 g/mL); 8: a second band for green garlic (30-45%, densities 1.12-1.18 g/mL); 9: a first band for ginger (8-30%, densities 1.02-1.12 g/mL); 10: a first band for turmeric (8-30%, densities 1.02-1.12 g/mL); 11: a second band for turmeric (30-45%, densities 1.12-1.18 g/mL); 12: a first band for shequasar (8-30%, densities 1.02-1.12 g/mL); 13: a second band for shequasar (30-45%, densities 1.12-1.18 g/mL); 14: a first band for aloe (8-30%, densities 1.02-1.12 g/mL); 15: a second band for aloe (30-45%, densities 1.12-1.18 g/mL); 16: a first band for grapefruit (8-30%, densities 1.02-1.12 g/mL); 17: a second band for grapefruit (30-45%, densities 1.12-1.18 g/mL); 18: a second band for crimson glory vine (30-45%, densities 1.12-1.18 g/mL); 19: a first band for Oriental melon (8-30%, densities 1.02-1.12 g/mL); 20: a first band for golden shequasar (8-30%, densities 1.02-1.12 g/mL); 21: a second band for golden shequasar (30-45%, densities 1.12-1.18 g/mL); 22: a first band for green papaya (8-30%, densities 1.02-1.12 g/mL); 23: a second band for green papaya (30-45%, densities 1.12-1.18 g/mL); and 24: a second band for bitter melon (30-45%, densities 1.12-1.18 g/mL).

As can be seen from the figure, the extracellular vesicles prepared from coastal hog fennel, Madeira vine, lemongrass, green garlic, turmeric, crimson glory vine, and bitter melon strongly suppressed the LPS-induced expression of IL-6, IL-1β, and IL-33. The extracellular vesicles from aloe suppressed the LPS-induced expression of IL-1B and IL-33, while those from Oriental melon suppressed the LPS-induced expression of IL-6 and IL-33. No extracellular vesicle suppressed the LPS-induced expression of TNF-α.

### (10) Effects of Small RNAs

Small RNAs were extracted from extracellular vesicles derived from coastal hog fennel using Direct-zol RNA MicroPrep (ZYMO RESEARCH), and the quality of the small RNAs thus obtained was examined using BioAnalyzer (Agilent Technologies). Subsequently, a library was prepared using NEB Next® Multiplex Small RNA Library Prep Set for Illumina® (Set 1) (Cat No. E7300, New England Biolabs). The resulting library was sequenced using NovaSeq 6000 (Illumina) as 100 bp single reads. The reads were trimmed to a read length of 10-40 bp using Trim Galore to remove adapter sequences and poly A sequences, and used as sequence data of the library.

On the other hand, miRNAs having the following sequences were chemically synthesized. The hsa numbers denote sequences of human-derived miRNA, while the mmu numbers denote sequences of mouse-derived miRNA.

hsa-miR-6737-3p:
(SEQ ID NO: 1)
UCUGUGCUUCACCCCUACCCAG
mmu-miR-7006-3p:
(SEQ ID NO: 2)
UUUCUGACCUGGAUCCCCAG
mmu-miR-6995-3p:
(SEQ ID NO: 3)
UGUGUCCCCUUCCUCUCACAG
mmu-miR-12178-5p:
(SEQ ID NO: 4)
UAUGGGUGGCUGGUCUCAGAAGAG
mmu-miR-3063-3p:
(SEQ ID NO: 5)
UGAGGAAUCCUGAUCUCUCGCC
mmu-miR-487b-5p:
(SEQ ID NO: 6)
UGGUUAUCCCUGUCCUCUUCG

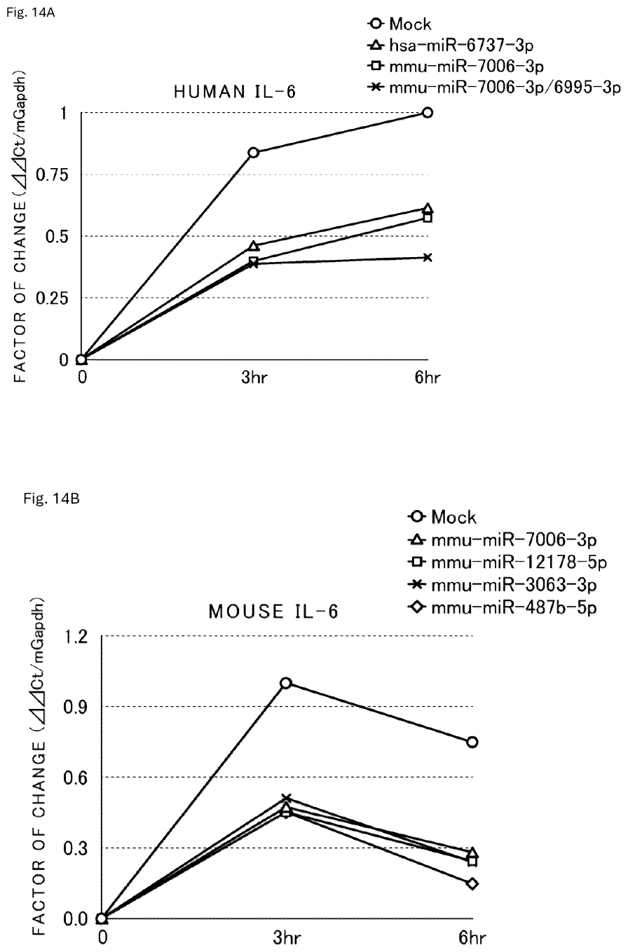

Next, human macrophage-derived THP-1 cells were transfected with miRNAs using Lipofectamine RNAiMAX (Thermo Fisher). After 24 hours, the cells were stimulated with 1 μg/ml LPS for 0, 3, or 6 hours (0 hours=no stimulation), and the expression of IL-6 was measured using RT-qPCR. As a result, the expression of IL-6 was suppressed when has-mira-6737-3p, mmu-miR-7006-3p, and mmu-miR-7006-3p were co-expressed with mmu-miR-6995-3p (FIG. 14A).

On the other hand, mmu-miR-487b-5p, mmu-miR-3063-3p, mmu-miR-7006-3p, and mmu-miR-12178-5p were expressed in mouse macrophage-derived RawBlue cells and stimulated with LPS. Then, the expression of IL-6 was measured. As a result, all of the miRNAs suppressed the LPS-induced expression of IL-6 (FIG. 14B).

Thus, the miRNAs indicated above are useful as suppressors of the IL-6 expression.

Note that these sequences were present in the sequence data of the small RNA library obtained from the extracellular vesicles derived from coastal hog fennel in an acceptable mismatch of two bases outside the seed sequence. The small RNAs derived from coastal hog fennel have the following sequences:

Pj-has-miR-6737-3p:
(SEQ ID NO: 23)
UCUG(a/c)GCU(a/g)CACCCCUACCCAG
Pj-mmu-miR-7006-3p:
(SEQ ID NO: 24)
UUUCUGAgCUGcAUCCCCAG
Pj-mmu-miR-6995-3p:
(SEQ ID NO: 25)
UGUGUCCCCaUCgUCUCACAG
Pj-mmu-miR-12178-5p:
(SEQ ID NO: 26)
UAaGGGUGGCUGtUCUCAGAAGAG
Pj-mmu-miR-3063-3p:
(SEQ ID NO: 27)
aGgGGAAUCCUGAUCUCUCGCC
Pj-mmu-miR-487b-5p:
(SEQ ID NO: 28)
UGGUUAUCCCUccCCUCUUCG

The small RNAs derived from coastal hog fennel have high homology to the aforementioned mammalian miRNAs. Accordingly, it is considered that they have an effect of suppressing the LPS-stimulated expression of IL-6.

1. A pharmaceutical composition comprising an effective amount of extracellular vesicles derived from a plant.

2. A pharmaceutical product comprising a pharmaceutical composition of claim 1.

3. The pharmaceutical product according to claim 2, wherein the plant is selected from the group consisting of coastal hog fennel, Madeira vine, lemongrass, green garlic, turmeric, crimson glory vine, and bitter melon.

4. The pharmaceutical product according to claim 2, wherein the pharmaceutical product is an anti-inflammatory agent, a muscle strength enhancer, a therapeutic agent for diabetes, a prophylactic agent for diabetes, an agent for reducing subcutaneous fat, a slimming agent, a therapeutic agent for dry eye syndrome, a therapeutic agent for irritable bowel syndrome, a cytokine storm inhibitor, a therapeutic agent for coronavirus infection, or a therapeutic agent for dementia.

5. The pharmaceutical product according to claim 4, wherein the anti-inflammatory agent is a therapeutic agent for encephalitis, myalgic encephalomyelitis, chronic fatigue syndrome, post-coronavirus diseases, inflammatory bowel diseases or dermatitis.

6. The pharmaceutical product according to claim 2, wherein the pharmaceutical product is an ointment, an oral medicament, eye drops, nasal drops, an inhalant, or an injection.

7. The pharmaceutical product according to claim 2, wherein an average particle diameter of the extracellular vesicles is in the range of 125-155 nm, 135-165 nm, 110-140 nm, or 160-190 nm.

8. The pharmaceutical product according to claim 2, wherein an average particle diameter of the extracellular vesicles is in the range of 135-145 nm, 145-155 nm, 120-130 nm, or 170-180 nm.

9. The pharmaceutical product according to claim 2, wherein 90% or more of the extracellular vesicles have a particle diameter in the range of 50-250 nm.

10. The pharmaceutical product according to claim 2, wherein 95% or more of the extracellular vesicles have a particle diameter in the range of 50-250 nm.

11. A food composition comprising an effective amount of extracellular vesicles derived from a plant.

12. A food product comprising the food composition of claim 11.

13. The food product according to claim 12, wherein the plant is selected from the group consisting of coastal hog fennel, Madeira vine, lemongrass, green garlic, turmeric, crimson glory vine, and bitter melon.

14. The food product according to claim 12, wherein an average particle diameter of the extracellular vesicles is in the range of 125-155 nm.

15. (canceled)

16. The food product according to claim 12, wherein 90% or more of the extracellular vesicles have a particle diameter in the range of 50-250 nm.

17. (canceled)

18. A cosmetic composition comprising an effective amount of extracellular vesicles derived from a plant.

19. A cosmetic product comprising the cosmetic composition of claim 18.

20. The cosmetic product according to claim 19, wherein the plant is selected from the group consisting of coastal hog fennel, Madeira vine, lemongrass, green garlic, turmeric, crimson glory vine, and bitter melon.

21. The cosmetic product according to claim 19, wherein an average particle diameter of the extracellular vesicles is in the range of 125-155 nm.

22. (canceled)

23. The cosmetic product according to claim 19, wherein 90% or more of the extracellular vesicles have a particle diameter in the range of 50-250 nm.

24.-31. (canceled)
